(19) United States
(12) Patent Application Publication
(10) Pub. No.: US 2005/0031683 A1
(43) Pub. Date: Feb. 10, 2005

(54) Solid pharmaceutical composition

(76) Inventors: Ashish Kapoor, Hyderabad (IN); Kolakaluri Uma Maheswara Prasad, Hyderabad (IN); Billa Praveen Reddy, Hyderabad (IN); Vijay Dinanathji Nasare, Hyderabad (IN); Mailatur Sivaraman Mohan, Hyderabad (IN)

Correspondence Address: DR. REDDY'S LABORATORIES, INC., 200 Somerset Corporate Blvd, SEVENTH FLOOR,, BRIDGEWATER, NJ, 08807-2862, US

(21) Appl. No.: 10/910,952

(22) Filed: Aug. 4, 2004

(30) Foreign Application Priority Data
Aug. 4, 2003 (IN) 637/CHE/2003

(51) Int. Cl. A61K 31/496; A61K 31/47; A61K 9/20; A61K 9/28

(52) U.S. Cl. 424/464; 514/253.08; 514/312

(57) ABSTRACT

Tablets containing an 8-alkoxyquinolonecarboxylic acid antibacterial drug are prepared by: mixing the drug with polyethylene glycol; adding an excipient comprising at least one of a diluent, a disintegrant, a glidant, or a lubricant; and compressing the mixture into a tablet.

### INTRODUCTION TO THE INVENTION

The present invention relates to a pharmaceutical composition, more particularly to a tablet composition that incorporates a drug substance, and to a method of preparation of the tablet composition.

Direct compression, dry granulation and wet granulation are techniques widely used in pharmaceutical industry for tablet preparation. Direct compression is a most convenient method and least time consuming process, typically involving the physical mixing of an active ingredient and excipient substances, adding a lubricant and further blending to coat the mixture, then compressing the lubricated mixture directly using a tablet compression machine.

An alternative to direct compression is dry granulation, involving a formation of large compacted masses using slugging or compaction, then breaking the masses into smaller granules having a desired size, mixing with a lubricant, and finally compressing into tablets. Dry granulation is a relatively time-consuming process and involves compacting, breaking, lubrication and compression in stepwise manner.

Wet granulation is a method that involves granulating a dry mixture of an active ingredient and at least one pharmaceutically acceptable excipient, using a suitable fluid granulating mixture, sifting the wet mass, drying the wet mass, and sifting or milling the dried mixture to yield granules having the desired size, strength, compressibility and flow characteristics, lubricating the dried granules, and finally compressing lubricated granules into tablets. The entire procedure is quite tedious and involves numerous unit operations, which makes the process expensive.

The direct compression method is the easiest method for tablet manufacturing, but many active ingredients do not possess all of the properties such as compressibility and flow characteristics, which are required for direct compression. Moreover, many active ingredients show problems such as adhering to punches and/or dies during tableting. The tableting of certain active ingredients therefore becomes difficult due to their inherent properties or characteristics. Hence, it is highly desired to develop formulations comprising excipients, which will eliminate the problems like sticking, picking, poor flow and compressibility, making tableting possible by the direct compression.

Ducatman et al., in U.S. Pat. No. 4,686,212, report the preparation of a stable tablet composition of sodium aspirin. The tablets are prepared by mixing the drug with a polyethylene glycol-dibasic calcium phosphate or a polyethylene glycol-lactose binder, and an animal or vegetable oil lubricant, then subjecting the mixture to compression.

U.S. Pat. No. 4,793,999 to Sheth describes a method of manufacturing a pharmaceutical composition that has enhanced drug bioavailability, by mixing triamterene, hydrochlorothiazide, filler, and a disintegrant with a polyethylene glycol having a melting point about 55° C., heating to the glycol melting point and stirring only long enough for the components to fuse, then cooling by liquid nitrogen addition to form granules. The granules were then mixed with other desired excipient ingredients and compressed into tablets.

U.S. Pat. No. 5,534,534 to Makino et al. describes a formulation of an angiotensin II receptor antagonist drug that typically degrades following tableting, wherein an “oily substance” is mixed with the active and other ingredients, then the mixture is compressed into tablets. A suitable oily substance is polyethylene glycol.

Fluoroquinolones are broad-spectrum antibacterial agents, widely used to treat a variety of infections. The 8-alkoxyquinolonecarboxylic acid category of antibacterials includes gatifloxacin, (±)-1-cyclopropyl-6-fluoro-1,4-dihydro-8-methoxy-7-(3-methyl-1-piperazinyl)-4-oxo-3-quinolinecarboxylic acid, and moxifloxacin, 1-cyclopropyl-7-{S,S)-2,8-diazabicyclo[4.3.0]non-8-yl}-6-fluoro-8-methoxy-1,4-dihydro-4-oxo-3-quinoline carboxylic acid. Gatifloxacin, in the form of the racemic sesquihydrate, and moxifloxacin, in the form of a hydrochloride, are both now being sold in solid and liquid dosage forms.

U.S. Pat. No. 6,291,462 to Bartholomaeus et al. describes a pharmaceutical dosage form of gatifloxacin and its pharmaceutically acceptable salts or hydrates, having a multi-phase structure consisting of an inner phase and outer phase. The inner phase includes gatifloxacin and fillers, binders and disintegration aids, and the outer phase includes at least one disintegration aid and a lubricant. The formulation procedure includes blending of inner phase ingredients followed by granulation using a granulating liquid, drying, sieving, and mixing with the outer phase ingredients. These two-phase granules can then be tableted.

The gatifloxacin tableting process described in the Bartholomaeus et al. patent is time-consuming and involves numerous unit operations. However, the resulting product was reported to have beneficial disintegration properties: tablets made according to the invention reproducibly disintegrated in times ranging from 6.5 to 25 minutes, while tablets of the same hardness prepared by wet granulating the inner phase ingredients and lubricating with magnesium stearate disintegrated over periods of 3 minutes to 10 hours, and gave highly variable drug release profiles.

It would be desirable to have a more simple procedure for preparing tablets, which reduces the required number of unit operations and provides a product having desirable properties.

### SUMMARY OF THE INVENTION

The invention includes a method for preparing solid dosage forms containing a drug, such as an 8-alkoxyquinolonecarboxylic acid antibacterial drug, and further includes the prepared solid dosage forms.

In one aspect the invention includes a method for producing tablets comprising an 8-alkoxyquinolonecarboxylic acid antibacterial drug, comprising the steps of: (a) mixing the 8-alkoxyquinolonecarboxylic acid drug with polyethylene glycol; (b) adding at least one excipient comprising at least one of a diluent, a disintegrant, a glidant, or a lubricant; and (c) compressing the mixture into tablets.

In another aspect, the invention includes a method for producing tablets comprising a drug substance, comprising the steps of: (a) sifting a drug substance through a sieve having openings less than about 60 mesh; (b) mixing particles that pass through the sieve with a pharmaceutically acceptable excipient comprising at least one of a filler, a disintegrant, a glidant, a lubricant, and a binder; (c) granulating the mixture of (b) with a solution comprising at least one binder, optionally further including a fluid silicone substance; (d) mixing particles of the drug substance that were retained on the sieve during step (a) with a pharmaceutically acceptable excipient comprising at least one of a filler, a disintegrant, a glidant, a lubricant and a binder, then granulating using a granulating solution; and (e) combining wet mixtures obtained from (c) and (d) and sifting, drying, and compressing into tablets.

In a further aspect, the invention includes a method for producing tablets comprising a drug substance, comprising the steps of: (a) sifting a drug substance through a sieve having openings less than, or equal to, about 60 mesh; (b) mixing particles passing through the sieve with a pharmaceutically acceptable excipient comprising at least one of a filler, a disintegrant, a glidant, a lubricant, or a binder; (c) granulating the mixture of (b) using a granulating solution comprising at least one binder, optionally further comprising a fluid silicone substance; (d) mixing particles of the drug substance retained on the sieve during step (a) with a pharmaceutically acceptable excipient comprising at least one of a filler, a disintegrant, a glidant, a lubricant, or a binder; and (e) combining mixtures obtained from (c) and (d), granulating using a granulating solution, drying, and compressing into tablets.

In a still further aspect, the invention includes a method for producing tablets, comprising the steps of: (a) sifting gatifloxacin powder through a sieve having openings less than, or equal to, about 100 mesh; (b) mixing particles passing through the sieve with a pharmaceutically acceptable excipient comprising at least one of a filler, a disintegrant, a glidant, a lubricant, or a binder; (c) granulating the mixture of (b) using about 80 volume percent of a granulating solution comprising at least one binder; (d) mixing particles of the drug substance retained on the sieve during step (a) with a pharmaceutically acceptable excipient comprising at least one of a filler, a disintegrant, a glidant, a lubricant, or a binder; and (e) combining mixtures obtained from (c) and (d), granulating using remaining granulating solution from (c), drying, and compressing into tablets.

### DETAILED DESCRIPTION OF THE INVENTION

The inventors have attempted to prepare gatifloxacin tablets, using various permutations of standard techniques for mixing the ingredients, adding lubricants, further mixing, and direct compression. However, in each attempt the tableting operation could not be successfully accomplished, due to severe sticking in the die and the formation of many tablets that exhibited capping. Through continued experimentation, the present invention was made.

Drug substances that can be formulated using the process of this invention include the 8-alkoxyquinolonecarboxylic acids, including their racemic mixtures, purified enantiomers, salts, esters, hydrates and other pharmaceutically acceptable derivatives. The two most important members of this class are gatifloxacin and moxifloxacin, both of which are very difficult to incorporate into tablets using the standard techniques and excipients. In general, the conditions for making acceptable moxifloxacin tablets using the invention are similar to those for gatifloxacin, so the following discussion and examples should be considered as applying to both drugs. Further, the present technique can be used for preparing tablets containing other drugs, whether or not those drugs might be successfully tableted using other, more standard processes and ingredients. Mixtures of 8-alkoxyquinolonecarboxylic acid antibacterial drug substances and other pharmaceutically active agents also can advantageously be tableted according to the present invention.

A representative pharmaceutical composition of said invention comprises gatifloxacin, including its enantiomers, and pharmaceutically acceptable salts, esters, hydrates, etc., optionally a polyethylene glycol, and at least one pharmaceutically acceptable excipient, such as a filler or diluent, disintegrant, glidant, binder, lubricant, etc.

Polyethylene glycols having the formula H(OCH₂CH₂)ₙOH are readily commercially available from many suppliers, the various products having average values of n from 4 to 160,000. For purposes of this invention, it generally is preferred to use polyethylene glycols having average molecular weight values between about 100 and 20,000, more preferably between about 200 and 8,000; the typical specifications for some commercial products falling within this preferred range are given in the table below:

| | | | | Melting |
|---|---|---|---|---|
| Product | Av. Molecular Wt. | Appearance | OH Value | Point (° C.) |
| PEG-200 | 190-210 | Oily liquid | 534-590 | <20 |
| PEG-300 | 285-315 | Oily liquid | 356-392 | <20 |
| PEG-400 | 380-420 | Oily liquid | 268-294 | <20 |
| PEG-600 | 570-630 | Oily liquid | 178-196 | 17-22 |
| PEG-1000 | 950-1050 | Solid | 108-117 | 35-40 |
| PEG-4000 | 3800-4400 | Solid | 22.5-29.5 | 53-58 |
| PEG-6000 | 5600-6400 | Solid | 17.5-20 | 55-60 |
| PEG-8000 | 7500-8500 | Solid | 13-15 | 58-65 |

Many excipients for tablet formation are known to those skilled in the art. Useful diluents or fillers include lactose, mannitol, sorbitol, starches, cellulose, dicalcium phosphate, calcium carbonate, compressible sugars, and others. Disintegrants include various starches, clays, celluloses, algins, gums, and cross-linked polymers; many derivatives of these more general substances are quite useful, including sodium starch glycolate, crospovidone, and croscarmellose. The glidants include substances such as colloidal silicon dioxide and talc. Binders include starch, gelatin, sugars, gums, cellulose derivatives, and others. Useful lubricants include magnesium stearate, stearic acid, a hydrogenated vegetable oil, a mineral oil, sodium lauryl sulfate, and others. It sometimes is beneficial to further incorporate one or more excipients such as a dye, a sweetening agent, a wetting agent such as sodium lauryl sulfate or a polysorbate, or a pH controlling agent such as citric acid, sodium citrate, monobasic sodium phosphate, magnesium oxide, etc.

In a preferred embodiment of the method of this invention, gatifloxacin and polyethylene glycol, and optionally a disintegrant, are mixed uniformly. After blending, one or more pharmaceutical diluents, such as microcrystalline cellulose, lactose, starch, or dicalcium phosphate is added. Optionally, a disintegrant is mixed with the blend of the above ingredients; examples of specific substances that are useful include sodium starch glycolate, croscarmellose sodium, low-substituted hydroxypropyl cellulose, sodium starch fumarate, cross-linked povidone, alginic acid, corn starch, and potato starch. Further, pharmaceutically acceptable lubricants and flow improvers are added to the dry blend. Lubricants such as magnesium or calcium stearate, stearic acid and the like are added and the blend is finally compressed into tablet.

In one preferred embodiment of an invention, the composition comprises, on a weight basis, about 40-50 percent active drug ingredient, and polyethylene glycol comprises 0.5 to 5 percent, preferably 0.5 to 3 percent, and more preferably 1 to 1.5 percent.

The invention also provides a preferred method for tableting active ingredients, which have processing problems during dry granulation and direct compression. In the method, an active ingredient is mixed with polyethylene glycol and optionally a disintegrant. After blending for a sufficient time, other pharmaceutically active ingredients, diluents, disintegrants, glidants, and lubricants are added, and the lubricated blend is compressed into tablets. The process does not have problems such as sticking, picking, and lamination.

In another preferred embodiment of an invention tablets are prepared by a wet granulation technique, avoiding a separate unit operation of lubrication after drying; the entire dry blend containing lubricants is granulated, then dried and compressed into tablets. A useful procedure for wet granulation tableting is as follows:
1. An active ingredient is first sifted through a sieve having openings less than or equal to about 60 mesh;
2. Particles of active material that pass through the sieve are mixed with one or more pharmaceutically acceptable lubricants, such sodium stearyl fumarate, optionally in combination with a solid polyethylene glycol, and this dry blend is mixed with at least one of a pharmaceutically acceptable filler, a disintegrant, a flow improver or glidant, a lubricant, and optionally a binder;
3. A suitable granulating solution (aqueous or nonaqueous) comprising at least one binder, either alone or in combination with a fluid silicone substance such as dimethicone, cyclomethicone, etc., is prepared and the lubricated mass is granulated using about 80 percent of the prepared granulating solution;
4. Material that is retained on the sieve during step one, at least one pharmaceutically acceptable filler, a disintegrant, a flow improver or glidant, a lubricant and optionally a binder and a polyethylene glycol are mixed, then granulated using the remaining 20 percent of the granulating solution; and
5. The wet masses from steps 3 and 4 are mixed, sifted, dried, and compressed into tablets.

After tablets have been formed, they can be protected against chipping, abrasion, or the adverse effects of humidity during storage, and their appearance can be improved, by applying a sugar or film coating. Techniques and materials for applying such coatings are well known in the art. Since sugar coating is a slow, labor-intensive operation, film-coating is usually preferred, using polymers such as cellulose acetate phthalate, hydroxypropyl methylcellulose, hydroxypropyl cellulose, methacrylate-methyl methacrylate copolymers, polyvinyl alcohol, and others. Compositions for applying film coatings can be purchased from a number of sources, and typically are complete systems containing the polymer, a plasticizer, and any desired pigments; after mixing with a suitable solvent for the polymer, such as water or an alcohol, the coatings can be applied by spraying, in a fluidized bed, or by any other known technique.

Further, in instances where it is desired to delay release of the drug from a dosage form so that the release occurs at some point after the dosage form passes through the stomach, an enteric coating can be applied to the tablet. These are applied using well-known techniques and contain acid-resistant polymers such as cellulose acetate phthalate, polyvinyl acetate phthalate, methacrylic acid-methacrylic acid ester copolymers, hydroxypropyl methylcellulose phthalate, hydroxypropyl methylcellulose succinate, carboxymethyl ethylcellulose, and others. Many useful enteric coating systems are commercially available.

The tablets prepared according to any of the examples below generally will disintegrate within about 5 minutes in 0.1N HCl, and release at least about 75 percent of the contained drug into 0.1N HCL within 30 minutes, when tested for drug solubility characteristics according to Method 711 of The United States Pharmacopeia 24, United States Pharmacopeial Convention, Inc., Rockville, Md. USA, 2000.

The following examples, while illustrating the invention and demonstrating certain embodiments, are not intended to limit the scope of the invention as defined by the appended claims, but serve only to represent some ways in which the invention can be carried out. In the examples, the gatifloxacin, used as the racemic sesquihydrate, was a powder wherein 90 percent by weight of the particles have sizes less than or equal to 400 μm.

### EXAMPLE 1

Tablets weighing 850 mg were prepared, using the following:

| Ingredients | mg/Tablet |
|---|---|
| Gatifloxacin | 400 |
| Polyethylene glycol 6000 | 5 |
| Sodium starch fumarate | 8.5 |
| Microcrystalline cellulose | 386.5 |
| Croscarmellose sodium | 40 |
| Colloidal silicon dioxide | 5 |
| Magnesium stearate | 8.5 |

Gatifloxacin was mixed with polyethylene glycol 6000 and sodium starch fumarate for up to 45 minutes. Microcrystalline cellulose and croscarmellose sodium were added to the dry blend and mixed uniformly. Further, magnesium stearate and colloidal silicon dioxide were added to the above blend and mixed. Finally, the blend was compressed into tablets. The tableting proceeded smoothly, without problems of material sticking to the die.

### EXAMPLE 2

Tablets weighing 850 mg were prepared, using the following:

| Ingredients | mg/Tablet |
|---|---|
| Gatifloxacin | 400 |
| Polyethylene glycol 6000 | 10 |
| Microcrystalline cellulose | 386.5 |
| Croscarmellose sodium | 40 |
| Colloidal silicon dioxide | 5 |
| Magnesium stearate | 8.5 |

Gatifloxacin was mixed with polyethylene glycol 6000 for up to 45 minutes. Microcrystalline cellulose and croscarmellose sodium were added to the dry blend and mixed uniformly. Magnesium stearate and silicon dioxide were added to the the blend and mixed. Finally, the blend was compressed into tablets. The tableting proceeded smoothly, without problems of material sticking to the die.

### EXAMPLE 3

In another embodiment of the invention, 850 mg tablets were prepared from the following:

| Ingredients | mg/Tablet |
|---|---|
| Gatifloxacin | 400 |
| Polyethylene glycol 400 | 10 |
| Microcrystalline cellulose | 386.5 |
| Croscarmellose sodium | 40 |
| Colloidal silicon dioxide | 5 |
| Magnesium stearate | 8.5 |

Gatifloxacin was mixed with the polyethylene glycol. The mixture was then sifted and blended uniformly with microcrystalline cellulose and croscarmellose sodium, added in a step-wise manner. After uniformly mixing all the ingredients above, magnesium stearate and silicon dioxide were added and blended. Finally, the mixture was compressed into tablets. No sticking of powder to the die was noted.

### EXAMPLE 4

A wet granulation method was used to prepare tablets containing either 200 or 400 mg of gatifloxacin, using the following:

| | 400 mg | | 200 mg | |
|---|---|---|---|---|
| | Tablets | | Tablets | |
| | mg/Tablet | | mg/Tablet | |
| Ingredients | Part 1 | Part 2 | Part 1 | Part 2 |
| Tablet | | | | |
| Gatifloxacin anhydrous | 340 | 60 | 170 | 30 |
| Sodium stearyl fumarate | 1 | 4 | 0.5 | 2 |
| Polyethylene glycol 6000 | 1 | 5 | 0.5 | 2.5 |
| Talc | 1 | 15 | 0.5 | 7.5 |
| Colloidal silicon dioxide | 4 | 4 | 2 | 2 |
| Microcrystalline cellulose | 15 | 329.5 | 7.5 | 164.75 |
| Croscarmellose sodium | 40 | 5 | 20 | 2.5 |
| Sodium stearyl fumarate | — | 8 | — | 4 |
| Magnesium stearate | 0.5 | 4 | 0.25 | 2 |
| Povidone | 9.6 | 2.4 | 4.8 | 1.2 |
| Dimethicone | 0.8 | 0.2 | 0.4 | 0.1 |
| Total weight | 412.9 | 437.1 | 206.45 | 218.55 |

| | | |
|---|---|---|
| Tablet weight | 850 | 425 |
| Film coating | | |
| Opadry yellow 03B52744 | 21.25 | 10.63 |
| Cum. tablet weight | 871.25 | 435.63 |

Gatifloxacin powder was sieved through 100 mesh openings, and a Part 1 mixture was made by mixing the undersized gatifloxacin particles, polyethylene glycol, talc, colloidal silicon dioxide, microcrystalline cellulose, croscarmallose sodium, and magnesium stearate in a rapid mass granulator. The dry mix thus obtained was granulated with 80 percent by volume of a granulating solution prepared by dissolving povidone and dispersing dimethicone in isopropyl alcohol.

Further, a Part 2 mixture of the gatifloxacin particles retained on the 100 mesh sieve was made by blending with the first listed amount of sodium stearyl fumarate and the polyethylene glycol 6000, then talc, colloidal silicon dioxide, microcrystalline cellulose, magnesium stearate, croscarmellose sodium were added, then the second listed amount of sodium stearyl fumarate was added and this dry blend was mixed with the above wet mass.

The total blended mass was then granulated with the remaining 20 percent of the granulating solution, sifted through a 12-mesh sieve and further dried in a fluid bed dryer to obtain a mixture having about a 4 percent by weight loss on drying, and then the dried granules were directly compressed into tablets. The tablets were film-coated with the Opadry™ product (obtained from Colorcon, West Point, Pa. USA), which is primarily a solution of hydroxypropyl methylcellulose in a solvent that is 40% isopropyl alcohol and 60% dichloromethane, plus a plasticizer and a pigment.

### EXAMPLE 5

Tablets produced in accordance with Example 4 and containing 400 mg of gatifloxacin were tested, using the apparatus and procedure of Method 711 (“Dissolution”) of The United States Pharmacopeia 24, United States Pharmacopeial Convention, Inc., Rockville, Md. USA, 2000. Using Apparatus 2 and a pH 4.5 buffered phosphate solution, the following results were obtained:

| | Percent Drug in Solution | | | | |
|---|---|---|---|---|---|
| Tablet | 0 | 15 | | | |
| No. | Minutes | Minutes | 30 Minutes | 45 Minutes | 60 Minutes |
| 1 | 0 | 90 | 92 | 94 | 93 |
| 2 | 0 | 90 | 91 | 91 | 91 |
| 3 | 0 | 90 | 91 | 93 | 94 |
| 4 | 0 | 91 | 92 | 92 | 92 |
| 5 | 0 | 88 | 89 | 90 | 90 |
| 6 | 0 | 94 | 94 | 94 | 95 |
| Mean | 0 | 90 | 92 | 92 | 92 |

Thus, it can be predicted that substantially all of the contained drug remaining after passage of a tablet through the stomach will be released within a very short time after the tablet enters the intestines. If the tablet had been enteric-coated with a film that resists acid, but which dissolves upon exposure to elevated pH conditions, then these results predict a prompt drug release after the tablet leaves the stomach.

1. A method for producing tablets comprising an 8-alkoxyquinolonecarboxylic acid antibacterial drug, comprising the steps of:
(a) mixing the 8-alkoxyquinolonecarboxylic acid drug with polyethylene glycol;
(b) adding an excipient comprising at least one of a diluent, a disintegrant, a glidant, or a lubricant; and
(c) compressing the mixture into tablets.

2. The method of claim 1, wherein the 8-alkoxyquinolonecarboxylic acid drug comprises gatifloxacin.

3. The method of claim 1, wherein the 8-alkoxyquinolonecarboxylic acid drug comprises moxifloxacin.

4. The method of claim 1, wherein the polyethylene glycol has an average molecular weight between about 200 and about 8,000.

5. The method of claim 1, wherein in step (b) the excipient comprises a diluent, a disintegrant, a glidant, and a lubricant.

6. A method for producing tablets comprising a drug substance, comprising the steps of:
(a) sifting a drug substance through a sieve having openings less than, or equal to, about 60 mesh;
(b) mixing particles passing through the sieve with a pharmaceutically acceptable excipient comprising at least one of a filler, a disintegrant, a glidant, a lubricant, or a binder;
(c) granulating the mixture of (b) using a granulating solution comprising at least one binder, optionally further comprising a fluid silicone substance;
(d) mixing particles of the drug substance retained on the sieve during step (a) with a pharmaceutically acceptable excipient comprising at least one of a filler, a disintegrant, a glidant, a lubricant, or a binder, then granulating using a granulating solution; and
(e) combining wet mixtures obtained from (c) and (d), drying, and compressing into tablets.

7. The method of claim 6, wherein the drug substance comprises an 8-alkoxyquinolonecarboxylic acid antibacterial drug

8. The method of claim 6, wherein the drug substance comprises gatifloxacin.

9. The method of claim 6, wherein the drug substance comprises moxifloxacin.

10. The method of claim 6, wherein the sieve of (a) has openings less than, or equal to, about 100 mesh.

11. The method of claim 6, wherein the excipient of (b) comprises a filler, a disintegrant, a glidant, a lubricant, and a binder.

12. The method of claim 6, wherein the excipient of (d) comprises a filler, a disintegrant, a glidant, a lubricant, and a binder.

13. The method of claim 6, wherein the granulating solutions of (c) and (d) have similar compositions and the amount of granulating solution used in (d) is about one-fourth of the amount used in (c).

14. A method for producing tablets comprising a drug substance, comprising the steps of:
(a) sifting a drug substance through a sieve having openings less than, or equal to, about 60 mesh;
(b) mixing particles passing through the sieve with a pharmaceutically acceptable excipient comprising at least one of a filler, a disintegrant, a glidant, a lubricant, or a binder;
(c) granulating the mixture of (b) using a granulating solution comprising at least one binder, optionally further comprising a fluid silicone substance;
(d) mixing particles of the drug substance retained on the sieve during step (a) with a pharmaceutically acceptable excipient comprising at least one of a filler, a disintegrant, a glidant, a lubricant, or a binder; and
(e) combining mixtures obtained from (c) and (d), granulating using a granulating solution, drying, and compressing into tablets.

15. The method of claim 14, wherein the drug substance comprises an 8-alkoxyquinolonecarboxylic acid antibacterial

16. The method of claim 14, wherein the drug substance comprises gatifloxacin.

17. The method of claim 14, wherein the drug substance comprises moxifloxacin.

18. The method of claim 14, wherein the sieve of (a) has openings equal to, or less than, about 100 mesh.

19. The method of claim 14, wherein the excipient of (b) comprises a filler, a disintegrant, a glidant, a lubricant, and a binder.

20. The method of claim 14, wherein the excipient of (d) comprises a filler, a disintegrant, a glidant, a lubricant, and a binder.

21. The method of claim 14, wherein the granulating solutions of (c) and (e) have similar compositions and the amount of granulating solution used in (e) is about one-fourth of the amount used in (c).

22. The method of claim 14, wherein the pharmaceutical excipient of (b) comprises a polyethylene glycol.

23. The method of claim 14, wherein the pharmaceutical excipient of (d) comprises a polyethylene glycol.

24. The method of claim 14, wherein the pharmaceutical excipients of (b) and (d) comprise a polyethylene glycol.

25. A method for producing tablets, comprising the steps of:
(a) sifting gatifloxacin powder through a sieve having openings less than, or equal to, about 100 mesh;
(b) mixing particles passing through the sieve with a pharmaceutically acceptable excipient comprising at least one of a filler, a disintegrant, a glidant, a lubricant, or a binder;
(c) granulating the mixture of (b) using about 80 volume percent of a granulating solution comprising at least one binder;
(d) mixing particles of the drug substance retained on the sieve during step (a) with a pharmaceutically acceptable excipient comprising at least one of a filler, a disintegrant, a glidant, a lubricant, or a binder; and
(e) combining mixtures obtained from (c) and (d), granulating using remaining granulating solution from (c), drying, and compressing into tablets.

26. The method of claim 25, wherein the pharmaceutical excipient of (b) comprises a polyethylene glycol.

27. The method of claim 25, wherein the pharmaceutical excipient of (d) comprises a polyethylene glycol.

28. The method of claim 25, wherein the pharmaceutical excipients of (b) and (d) comprise a polyethylene glycol.

29. A tablet prepared by the method of claim 1.

30. A tablet prepared by the method of claim 6.

31. A tablet prepared by the method of claim 14.

32. A tablet prepared by the method of claim 25.
